(19) United States
(12) Patent Application Publication
(10) Pub. No.: US 2026/0365607 A1
(43) Pub. Date: Nov. 19, 2026

(54) MANUFACTURE OF AN ETHYLENE-DERIVED CHEMICAL OF INTEREST, IN PARTICULAR ACRYLIC ACID, IN COMBINATION WITH GENERATION OF HEATED STEAM

(71) Applicant: BASF SE, Ludwigshafen am Rhein (DE)

(72) Inventors: Stefan WILLERSINN, Houston, TX (US); Christian WEINEL, Ludwigshafen am Rhein (DE); Daniel KECK, Ludwigshafen am Rhein (DE); Johannes Lazaros Friedrich ELLER, Ludwigshafen am Rhein (DE); Dieter RAHN, Ludwigshafen am Rhein (DE)

(21) Appl. No.: 19/141,077

(22) PCT Filed: Dec. 18, 2023
(86) PCT No.: PCT/EP2023/086350

§ 371 Date: Jun. 19, 2025

(30) Foreign Application Priority Data
Dec. 20, 2022 (EP) 22215079.9

(51) Int. Cl. C07C 57/04; C01B 32/50; C07C 1/24; C07C 2/06; C07C 5/25; C07C 6/04; C07C 11/04; C07C 11/06; C07C 11/08; C07C 51/16

(52) U.S. Cl. CPC C07C 57/04; C01B 32/50; C07C 1/24; C07C 2/06; C07C 5/2506; C07C 6/04; C07C 11/04; C07C 11/06; C07C 11/08; C07C 51/16

(57) ABSTRACT

A process for the manufacture of acrylic acid in combination with generation of heated steam comprises subjecting a feedstock comprising a renewably-sourced ethanol to dehydration to produce a renewably-sourced ethylene stream, which is subjected to an olefin-interconversion to obtain a renewably-sourced propylene. Further, the renewably-sourced propylene is subjected to an oxidation reaction to obtain acrylic acid. At least a portion of heat generated by the oxidation reaction is removed, and at least a portion of the heat is transferred to a water stream to generate a heated steam stream. The process provides a reaction scheme that provides renewably-sourced acrylic acid, while minimizing both the non-renewable fossil energy input and consumption of renewably-sourced feedstock.

### TECHNICAL BACKGROUND

The present invention relates to a process for the manufacture of an ethylene-derived chemical of interest, specifically the oxidation products of an olefin selected from ethylene and C₃₋₄-olefin, in combination with generation of heated steams.

Ethylene is a cornerstone of the modern petrochemical industries. Important ethylene derivatives (at the end of their respective chains) include (meth)acrylic acid, (meth)acrylic esters, isononanols, ethylhexanol, and ethylene glycols. One of the problems faced by the manufacture of chemicals and intermediates from ethylene is that the starting raw materials are from fossil fuels, such as natural gas or crude oil, which are non-renewable feedstocks. Steam cracking, which employs petroleum fractions and natural gas liquids as feedstocks, is the dominant method for large-scale ethylene production worldwide.

The production of the production of ethylene and ethylene derivatives compounds would benefit from the replacement of at least a part of the carbonaceous raw materials of fossil origin by renewable resources, such as carbonaceous matter derived from biomass. Of particular interest is the ethanol feedstock which is produced from renewable resources. Such renewably-sourced ethanol, also referred to as “bioethanol” or “hydrous fuel alcohol” can be prepared in large quantities from organic waste or biomass via fermentation. The different feedstocks for producing ethanol may be sucrose-containing feedstocks, e.g., sugarcane, starchy materials, e.g., corn, starch, wheat, cassava, lignocellulosic biomass, e.g., switchgrass, and/or agricultural waste.

Renewably-sourced ethanol can be converted into renewably-sourced propylene by a sequence of steps that includes ethanol dehydration to produce renewably-sourced ethylene and subjecting the renewably-sourced ethylene stream to an olefin-interconversion, to obtain renewably-sourced propylene. The olefin-interconversion, in turn, comprises ethylene dimerization to obtain n-butenes; and metathesis reaction between n-butenes and ethylene to obtain propylene.

Ethanol dehydration, however, is a highly endothermic reaction. Also the olefin conversion steps and the ensuing separation of material streams by distillation or similar processes require energy input. Hence, in assessing the overall environmental benefit the non-renewable fossil energy inputs used in producing the renewably-sourced feedstock and its conversion must be considered. While it could be envisaged to burn part of the bio-ethanol to generate thermal energy, the bio-ethanol used as a fuel for energy generation is no longer available as a feedstock for chemical product manufacture.

The economic feasibility of ethanol dehydrogenation and olefin conversion steps could be improved if the initial energy input could be at least partially offset by the recovery of green energy downstream the process. This would allow reducing energy costs by implementing heat integration design, that is, the process-wide pairing of heat-releasing streams with heat-accepting streams.

A number of important basic chemicals are manufactured by oxidation reactions of olefins over selective catalysts. For example, unsaturated aldehydes and carboxylic acids are obtained by allylic oxidation of olefins, while epoxides are obtained by epoxidation of olefins.

Owing to its very reactive double bond and the acid function, acrylic acid is suitable in particular for use as a monomer for preparing polymers. Of the overall quantity of acrylic acid monomer produced, the major part is esterified before polymerization, for example to form acrylate adhesives, dispersions or coatings. Only the smaller part of the acrylic acid monomer produced is polymerized directly, for example to form water-absorbent resins. Acrylic acid is industrially produced by heterogeneously catalyzed gas phase oxidation of propylene with molecular oxygen over solid catalysts in two stages via acrolein.

Ethylene oxide is used as a chemical intermediate, primarily for the production of ethylene glycols but also for the production of ethoxylates, ethanol-amines, solvents and glycol ethers. It is industrially produced by the direct oxidation of ethylene with oxygen or air.

US 2008/0312485 discloses a method for continuously producing propylene by dehydrating ethanol obtained from biomass to obtain ethylene and reacting ethylene with n-butene in a metathesis reaction. The n-butene is made by dimerization of ethylene which is obtained from biomass-derived ethanol.

WO 2009/098268 discloses a process for the dehydration of an alcohol to make an olefin. The alcohol may be ethanol that can be obtained from carbohydrates. For this purpose a stream comprising the ethanol and an inert component is contacted with a catalyst to yield ethylene. It is indicated that the ethylene can be used for dimerization to butene and then isomerization to isobutene, dimerization to 1-butene, which is isomerized to 2-butene and further converted by metathesis with ethylene to propylene, or conversion to ethylene oxide and glycol. Experimental details are provided only for ethanol dehydration. A similar process is disclosed in WO 2011/089235.

WO 2009/098269 discloses a process for conversion of ethanol that can be obtained from carbohydrates to propylene. The ethanol is dehydrated to ethylene which is reacted with olefins having four carbon atoms or more to give propylene. WO 2009/098267 discloses a similar process.

The invention seeks to advise a reaction scheme that provides renewably-sourced olefin oxidation products, while minimizing both the non-renewable fossil energy input and consumption of renewably-sourced feedstock.

### SUMMARY OF THE INVENTION

The invention relates to a process for the manufacture of an ethylene-derived chemical of interest in combination with generation of heated steam, said process comprising the steps of:
a) subjecting a feedstock comprising a renewably-sourced ethanol to dehydration to produce a renewably-sourced ethylene stream;
b) optionally, subjecting the renewably-sourced ethylene stream to an olefin-interconversion, to obtain a renewably-sourced C₃₋₄-olefin, selected from propylene, n-butenes and isobutene; the olefin-interconversion comprising (i) and, where required, one of (ii) and (iii):
   (i) ethylene dimerization to obtain n-butenes;
   (ii) metathesis reaction between n-butenes obtained according to (i) and ethylene to obtain propylene;
   (iii) isomerization of n-butenes obtained according to (i) to obtain isobutene; and
c) subjecting the renewably-sourced ethylene or renewably-sourced C₃₋₄-olefin to a chemical conversion or sequence of chemical conversions to obtain the chemical of interest;
wherein at least one of the chemical conversions is an exothermic chemical reaction;
removing at least a portion of heat generated by the exothermic chemical reaction and transferring at least a portion of the heat to a water stream to generate a heated steam stream.

In particular, the invention relates to a process for the manufacture of acrylic acid in combination with generation of heated steam, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion to obtain a renewably-sourced propylene; the olefin-interconversion comprising (i) and (ii):
   (i) ethylene dimerization to obtain n-butenes;
   (ii) metathesis reaction between n-butenes obtained according to (i) and ethylene to obtain propylene; and
c) subjecting the renewably-sourced propylene to an oxidation reaction to obtain acrylic acid; and
removing at least a portion of heat generated by the oxidation reaction and transferring at least a portion of the heat to a water stream to generate a heated steam stream.

While it is beneficial to use exclusively renewably-sourced ethylene or the renewably-sourced C₃₋₄-olefin in the chemical conversion or sequence of chemical conversions in step c), in an embodiment, the renewably-sourced ethylene or the renewably-sourced C₃₋₄-olefin is blended with a fossil-derived, e.g., petroleum-derived olefin of the same chemical structure. This may be an option for full plant utilization, periods of fluctuating raw material supply and/or for a transition period during the conversion to an exclusive use of renewably-sourced ethylene or the renewably-sourced C₃₋₄-olefin.

According to the invention, at least a portion of the heat energy produced by the exothermic chemical reaction is recovered by production of a heated steam stream Conveniently, the heated steam stream is used to provide heat to one or more heat-accepting processes. Modern industrial chemical production sites are integrated with multiple processes and plants to produce a variety of chemical products. A “heat accepting process” can be any process in an industrial chemical production site, that requires the input of thermal energy, typically provided by heated steam. A “heat accepting process” can be for instance any or all of steps a), b)-(i), b)-(ii) and b)-(iii) but is not limited thereto. A “distributed steam grid” is used for heat transfer between different processes within a site and thus the integration and effective use of heat. The heat energy can generate steam by vaporization of water, preferably in the form of steam condensate. The steam is fed into the steam grid. The heat of reaction can additionally be utilized in order to raise steam to a high pressure level, of 4 bar gauge or higher, as is typically provided in the steam lines in industrial plants. The steam can be utilized in equipment such as heat exchangers, steam turbines, reboilers, and the like.

In an embodiment, the heated steam stream is used to provide at least a portion of the heat consumed in at least one of steps a) and b), more specifically in at least one of steps a), b)-(i), b)-(ii) and b)-(iii). Thereby, the economic feasibility of ethanol dehydrogenation and olefin conversion steps can be improved because less fossil energy is required for steam generation.

Unfortunately, not every type of waste heat can be used economically. Heat recovered at a temperature too low for its convenient use in the plant, has to be disposed of with the cooling water, so energy-penalizing the overall process. Hence highly exothermic chemical reactions are preferred candidates for heat recovery by generation of heated steam. A reaction is considered exothermic if its standard enthalpy of reaction is negative. Preferably, the standard enthalpy of reaction (ΔH_{R}⁰) of the exothermic chemical reaction is less than −400 kJ/mol, more preferably in the range from −1500 to −450 kJ/mol, even more preferably in the range from −1400 to −500 kJ/mol.

The standard enthalpy of reaction

$$( Δ {H}_{R}^{0} )$$

is related to the standard enthalpy of formation

$$( Δ {H}_{f}^{0} )$$

of the reaction product(s) by the following equation:

$$Δ {H}_{R}^{0} = {\sum }_{products , p} Δ {H}_{f , p}^{0} - {\sum }_{reactants , r} Δ {H}_{f , r}^{0}$$

“Reactant(s)” mean(s) the chemical substance(s) that are subjected to the respective exothermic chemical reaction.

“Reaction product” means the reaction product that is directly obtained from the respective exothermic chemical reaction. It can be the chemical of interest or a respective intermediate along the chemical path to the chemical of interest.

The standard enthalpy of formation is the change of enthalpy during the formation of 1 mole of the substance (reactant or reaction product) from its constituent elements, with all substances in their standard states.

All enthalpy values refer to the standard conditions (25 °C and 1 bar).

In preferred embodiments, the exothermic chemical reaction is an oxidation reaction. In an oxidation reaction, a substrate is oxidized by an oxidant, preferably molecular oxygen or air, to obtain an oxidation product of the substrate. “Substrate” may be the renewably-sourced ethylene or renewably-sourced C₃₋₄-olefin (which is optionally blended with a fossil-derived olefin of the same chemical structure) or a conversion product thereof obtained by a chemical conversion or sequence of chemical conversions upstream of the oxidation reaction. The chemical conversions involved in the oxidation reactions are less than 100% selective. The yield losses manifest themselves in the over-oxidation of the substrate to form carbon oxides, in particular carbon dioxide. Over-oxidation of the substrate to form carbon dioxide contributes to the exothermic nature of the oxidation reaction. A considerable proportion of the heat released is generated by the complete oxidation of the olefinic substrate with the release of CO₂ resulting from the high enthalpy of reaction that is attributed to the full oxidation. If a renewably-sourced olefin is employed, this total oxidation to CO₂ can be regarded as a combustion of the renewably-sourced ethanol which is shifted to the oxidation reaction step. The key feature of this preferred embodiments is the specific combination of the renewably-sourced olefin production with the oxidation reaction of the respective renewably-sourced olefin. The steam thus generated can be regarded as “green steam” because the heat used for its generation can be attributed to the combustion of renewably-sourced ethanol, thus no fossil resources are required. Of course, one could also directly combust a certain amount of renewably-sourced ethanol (i.e. using it as a bio fuel) to generate the required heat. However, once combusted, the renewably-sourced ethanol could not be used anymore as a starting material for chemical synthesis. This amount of renewably-source ethanol can be saved by the specific combination according to this preferred embodiment. Put otherwise, the combustion of part of the renewably-sourced ethanol to generate heat is shifted to a later production step, namely the oxidation of the respective renewably-sourced olefin, as part of the inevitable yield losses there. Thus, this embodiment incorporates the use of renewably-sourced ethanol as a starting material for chemical synthesis and as a bio fuel at the same time.

Typically, in an oxidation reaction susceptible to heat recovery by generation of heated steam 1 to 10 mol.-%, preferably 2 to 8 mol.-% of the substrate is over-oxidized to carbon dioxide.

By a full or partial replacement of fossil ethylene and C₃₋₄-olefins by their renewably-sourced counterparts, the fossil-based carbon dioxide emissions can be reduced because the emissions resulting from yield losses are at least partially based on green carbon. The resulting carbon dioxide emissions therefore do not contribute to the green house emission of the production site. For example, in the production of acrylic acid or ethylene oxide (as further described below) carbon dioxide is formed due to full oxidation of propylene or ethylene, respectively. Using a renewably-sourced propylene or ethylene, respectively, in accordance with the present invention therefore prevents the formation of fossil-based carbon dioxide emissions resulting from such productions.

If renewably-sourced olefin is employed, the CO₂ formed is of biogenic origin and does not contribute to the greenhouse effect. The heat thus released has consequently not generated any CO₂ emissions.

In preferred embodiments of the process according to the invention, the amount of biogenic CO₂ released in the oxidation reaction is in the range from 50 to 100 wt.-%, preferably 80 to 100 wt.-%, based on the total amount of CO₂, i.e. including both biogenic CO₂ and fossil CO₂, released in the oxidation reaction.

Biogenic CO₂ may be distinguishable from fossil CO₂ on the basis of carbon-isotopic fingerprinting and/or ¹⁴C (fM). There are three naturally occurring isotopes of carbon: ¹²C, ¹³C, and ¹⁴C. These isotopes occur in above-ground total carbon at fractions of 0.989, 0.011, and 10~12, respectively. The isotopes ¹²C and ¹³C are stable, while ¹⁴C decays naturally to ¹⁴N with a half-life of 5730 years. The isotope ¹⁴C originates in the atmosphere, due primarily to neutron bombardment of ¹⁴N caused ultimately by cosmic radiation. Because of its relatively short half-life (in geologic terms), ¹⁴C occurs at extremely low levels in fossil carbon.

Alternatively, the ratio of fossil CO₂ to biogenic CO₂ may, of course, be assessed on the basis of the input of biogenic ethylene or C₃₋₄-olefins to fossil ethylene or C₃₋₄-olefins to the oxidation reaction.

Generally, the oxidation reaction is selected from allylic oxidation reactions and epoxidation reactions. As utilized herein, the term “allylic oxidation” means oxidation of an allylic compound by replacing allylic hydrogen(s) with oxygen or an oxygen containing group. Similarly, the term “epoxidation” means oxidation of an olefin by adding an oxygen atom to a carbon-carbon double bond to form an oxirane ring:

Preferably, the allylic oxidation reaction is selected from the oxidation of propylene to obtain acrolein and/or acrylic acid; and the oxidation of isobutene to obtain methacrolein and/or methacrylic acid.

Preferably, the epoxidation reaction is the epoxidation of ethylene to obtain ethylene oxide.

Generally, the oxidation reaction is a heterogeneously catalyzed gas-phase oxidation of with molecular oxygen. The oxidizing agent may be air, oxygen or other molecular oxygen containing gas. Air has the advantage of a lower cost but oxygen permits higher throughput per unit reactor volume.

The process of the invention is preferably a continuous process. This does not preclude the presence of buffer volumes between subsequent reaction steps in a reaction route.

The expressions “renewable” or “renewably-sourced” in relation to a chemical compound are used synonymously and mean a chemical compound comprising a quantity of renewable carbon, i.e., having a reduced or no carbon content of fossil origin. Renewable carbon entails all carbon sources that avoid or substitute the use of any additional fossil carbon from the geosphere. Renewable carbon can come from the biosphere, atmosphere or technosphere—but not from the geosphere. Thus, the expresssion “renewable” or “renewably-sourced” includes, in particular, biomass-derived chemical compounds. It also includes compounds derived from waste such as polymer residues, or from waste streams of chemical production processes.

Bioethanol is a preferred form of renewably-sourced ethanol, although the scope of the invention is not limited to the use of bioethanol.

In the present invention, bioethanol refers to the ethanol obtained from a biomass feedstock, such as plant or non-crop feedstock containing a carbon source that is convertible to ethanol, for example by microbial metabolism. Typical carbon source examples are starch, sugars like pentoses or hexoses, such as glucose, fructose, sucrose, xylose, arabinose, or degradation products of plants, hydrolysis products of cellulose or juice of sugar canes, beet and the like containing large amounts of the above components.

Biomass feedstock can originate from several sources. Bioethanol production may be based on food crop feedstocks such as corn and sugar cane, sugarcane bagasse, cassava (first generation biofeedstock).

Another source of biomass feedstock is lignocellulosic materials from agricultural crops (second-generation biofeedstock). Potential feedstocks include agricultural residue by-products such as rice, straw (such as wheat, oat and barley straw), rice husk, and corn stover. Biomass feedstock may also be waste material from the forest products industry (wood waste) and saw dust or produced on purpose as an ethanol crop. Switchgrass and napier grass may be used as on-purpose crops for conversion to ethanol.

The first-generation bioethanol is produced in four basic steps:
(1) enzymatic saccharification or hydrolysis of starch into sugars;
(2) microbial fermentation of sugars;
(3) purification by distillation to give hydrous ethanol; and
(4) dehydration (water removal) to produce anhydrous ethanol.

Second-generation feedstocks are considered as renewable and sustainable carbon source. Pretreatment of this feedstock is an essential prerequisite before it is subjected to enzymatic hydrolysis, fermentation, distillation, and dehydration. Pretreatment involves milling and exposure to acid and heat to reduce the size of the plant fibers and hydrolyze a portion of the material to yield fermentable sugars. Saccharification utilizes enzymes to hydrolyze another portion to sugar. Finally, fermentation by bioengineered microorganisms converts the various sugars (pentoses and hexoses) to ethanol. The production of bioethanol is well-known and carried out on an industrial large scale.

Renewably-sourced ethanol can also be obtained from carbon-containing waste materials like waste products from the chemical industry, garbage and sewage sludge. The production of ethanol from waste materials can be done by gasification to syngas and catalytic conversion thereof the ethanol, see for example Recent Advances in Thermo-Chemical Conversion of Biomass, 2015, Pages 213-250, https://doi.org/10.1016/B978-0-444-63289-0.00008-9, and Nat Commun 11, 827 (2020), https://doi.org/10.1038/s41467-020-14672-8.

### Dehydration of Renewably-Sourced Ethanol

As a first step, the invention involves the dehydration of renewably-sourced ethanol. The production of ethylene by catalytic dehydration of ethanol is a well-known process. The reaction is commonly carried out at 300 to 400 °C and moderate pressure in the presence of a catalyst. Catalytic effects are reviewed in Ind & Eng Chem Research, 52, 28, 9505-9514 (2013), Materials 6, 101-115 (2013) and ACS Omega, 2, 4287-4296 (2017). Examples for catalysts are activated alumina or silica, phosphoric acid impregnated on coke, heteropoly acids (HPA salts), silica-alumina, molecular sieves such as zeoliths of the ZSM-5 type or SAPO-11 type, other zeolites or modified zeolites of various molecular structures with zeoliths and HPA salts being preferred.

Ethanol dehydration is, for example described in WO 2009/098268, WO 2010/066830, WO 2009/070858 and the prior art discussed therein, WO 2011/085223 and the prior art discussed therein, U.S. Pat. Nos. 4,234,752, 4,396,789, 4,529,827 and WO 2004/078336.

The ethanol dehydration reaction is in general carried out in the vapor phase in contact with a heterogeneous catalyst bed using either fixed bed or fluidized bed reactors. For fixed bed reactors, the operation can be either isothermal (with external heating system) or adiabatic (in the presence of a heat carrying fluid). The feedstock is vaporized and heated to the desired reaction temperature; the temperature drops as the reaction proceeds in the reactor. Multiple reactor beds are usually used in series to maintain the temperature drop in each bed to a manageable range. The cooled effluent from each bed is further heated to bring it to the desired inlet temperature of the subsequent beds. Moreover, a portion of the water is recirculated along with fresh and unreacted ethanol. The presence of water helps in moderating the temperature decrease in each bed.

Prior to dehydration, the renewably-sourced ethanol feedstock may be sent to a pretreatment section to remove mineral contaminants, which would otherwise be detrimental to the downstream catalytic reaction. The pretreatment may involve contacting the renewably-sourced ethanol feedstock with cation and/or anion exchange resins. After a certain period of operation, the resins may be regenerated by passing a regenerant solution through the resin bed(s) to restore their ion exchange capacity. Two sets of beds are preferably operated in parallel to maintain continuous operation. One set of resin beds is suitably regenerated while the other set is being used for pretreatment.

In the isothermal design, the catalyst is placed inside the tubes of multitubular fixed-bed reactors which arranged vertically and surrounded by a shell (tube and shell design). A heat transfer medium, such as molten salts or oil, is circulated inside the shell to provide the required heat. Baffles may be provided on the shell side to facilitate heat transfer. The cooled heating medium is heated externally and is recirculated. The temperature drop on the process side can be reduced as compared to the adiabatic reactor. A better control on the temperature results in increased selectivity for the ethylene formation and reduction in the amount of undesireable by-products. The temperature is maintained at approximately constant levels within the range of 300° to 350 °C. Ethanol conversion is between 98 and 99%, and the selectivity to ethylene is between 94 and 97 mol %. Because of the rate of coke deposition, the catalyst must be regenerated frequently. Depending on the type of catalyst used, the cycle life is between 3 weeks and 4 months, followed by regeneration, for example for 3 days.

In the adiabatic design, the endothermic heat of reaction is supplied by a preheated inert diluent such as steam. Three fixed-bed reactors may typically be used, with intermediate furnaces to reheat the ethanol/steam mixed feed stream to each reactor. Feeding steam with ethanol results in less coke formation, longer catalyst activity, and higher yields.

A further process is a fluidized-bed process. The fluidized-bed system offers excellent temperature control in the reactor, thereby minimizing by-product formation. The heat distribution rate of the fluidized bed operation approaches isothermal conditions. The endothermic heat of reaction is supplied by the hot recycled silica-alumina catalyst returning from the catalyst regenerator. Thus, external heating of the reactor is not necessary.

After dehydration, the reaction mixture is subjected to a separation step. The general separation scheme consists of quickly cooling the reaction gas, for example in a water quench tower, which separates most of the by-product water and the unreacted ethanol from ethylene and other light components which, for example exit from the top of the quench tower. In one type of separation scheme, the water-washed ethylene stream is immediately caustic-washed, for example in a column, to remove traces of CO₂. The gaseous stream may enter a compressor directly or pass to a surge gas holder first and then to a gas compressor. After compression, the gas is cooled with refrigeration and then passed through an adsorber with, for example activated carbon, to remove traces of heavy components, (e.g., C4s), if they are present. The adsorber is followed by a desiccant drying and dust filtering step before the ethylene product leaves the plant. This separation scheme produces 99%+ purity ethylene. If desired, the ethylene is further purified by caustic washing and desiccant-drying, and fractionated in a low-temperature column to obtain the final product.

Several commercial processes are currently in operation, developed by Braskem, Chematur, British Petroleum (BP), and Axens together with Total and IFPEN. The processes differ, e.g., in their process conditions, catalysts and adopted heat integration scheme. The process by BP (now Technip) is called Hummingbird. In this process, a heteropoly acid is used as catalyst, and the reactor operates at 160 to 270 °C and 1 to 45 bar. The unreacted ethanol in recirculated to the reactor. The process developed by Axens is called Atol. Two fixed bed adiabatic reactors, operating at 400 to 500 °C, are used. Chematur's process operates with four adiabatic tubular reactors. Syndol catalysts, with the main components of Al₂O₃—MgO/SiO₂, are employed in this process that was developed by American Halcon Scientific Design, Inc. in the 1980s. In the Braskem process, the adiabatic reactor feed is diluted with steam to a large extent. In such a process, the reactor operates at 180 to 600 °C, preferably 300 to 500 °C, and at 1.9 to 19.6 bar. An alumina or silica-alumina catalyst is used. The Braskem process is described in more detail in U.S. Pat. No. 4,232,179. A process control in accordance with the Braskem process is particularly preferred.

### Dimerization of Ethylene

In one aspect, the process of the invention involves an ethylene-dimerization to obtain n-butenes in accordance with step b)-(i) above. Any known method can be used for ethylene dimerization to produce n-butenes. A review on dimerization and oligomerization chemistry and technology is given in Catalysis Today, vol. 14 (no. 1), Apr. 10, 1992.

Expediently, step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream; and
fractionating the effluent to recover a stream consisting essentially of n-butenes, a stream consisting essentially of heavier olefins, and an optional ethylene stream.

The dimerization catalyst may be homogeneous or heterogeneous. Typical dimerization catalysts are titanium or nickel compounds activated with alkyl aluminium compounds. In general, the Ti(IV) valency is stabilized by selecting the appropriate ligands, alkyl aluminium compound, the solvent polarity and the Al/Ti ratio. Nickel compounds that can catalyse the selective production of butenes are typically based on cationic nickel salts stabilised with phosphine and activated with alkyl aluminium compounds.

In one embodiment, the oligomerization of ethylene is implemented in the presence of a catalytic system in the liquid phase comprising a nickel compound and an aluminum compound. Such catalytic systems are described in the documents FR 2 443 877 and FR 2794 038. The Dimersol E™ process is based on this technology and leads to the industrial production of olefins.

Thus, in one embodiment, the oligomerization of ethylene is implemented in the presence of a catalytic system comprising:
i) at least one bivalent nickel compound,
ii) at least one hydrocarbyl aluminum dihalide of formula AIRX2, in which R is a hydrocarbyl radical comprising 1 to 12 carbon atoms, such as alkyl, aryl, aralkyl, alkaryl or cycloalkyl, X is a chlorine or bromine atom, and
iii) optionally a Brønsted organic acid.

As the bivalent nickel compound, nickel carboxylates of general formula (R¹COO)₂Ni are preferably used, where R¹ is an optionally substituted hydrocarbyl radical, for example alkyl, cycloalkyl, alkenyl, aryl, aralkyl, or alkaryl, containing up to 20 carbon atoms, preferably a hydrocarbyl radical of 5 to 20 carbon atoms, preferably 6 to 18 carbon atoms. Suitable bivalent nickel compounds include: chloride, bromide, carboxylates such as octoate, 2-ethylhexanoate, decanoate, oleate, salicylate, hydroxydecanoate, stearate, phenates, naphthenates, and acetyl acetonates. Nickel 2-ethylhexanoate is preferably used.

The hydrocarbyl aluminum dihalide compound corresponds to the formula AlRX₂, in which R is a hydrocarbyl radical comprising 1 to 12 carbon atoms, such as alkyl, aryl, aralkyl, alkaryl or cycloalkyl, and X is a chlorine or bromine atom. As examples of such compounds, it is possible to mention ethylaluminum sesquichloride, dichloroethyl aluminum, dichloroisobutyl aluminum, chlorodiethyl aluminum or mixtures thereof.

According to a preferred method, a Brønsted organic acid is used. The Brønsted acid compound corresponds to the formula HY, where Y is an organic anion, for example carboxylic, sulfonic or phenolic. Halocarboxylic acids of formula R²COOH in which R² is a halogenated alkyl radical are preferred, in particular those that contain at least one alpha-halogen atom of the group —COOH with 2 to 10 carbon atoms in all. Preferably, a haloacetic acid of formula CXₚH₃₋ₚ—COOH is used, in which X is fluorine, chlorine, bromine or iodine, with p being an integer from 1 to 3. By way of example, it is possible to cite the trifluoroacetic, difluoroacetic, fluoroacetic, trichloroacetic, dichloroacetic, and chloroacetic acids. It is also possible to use arylsulfonic, alkylsulfonic, and fluoroalkylsulfonic acids, and picric acid and nitroacetic acid. Trifluoroacetic acid is preferably used.

The three components of the catalytic formula can be mixed in any order. However, it is preferable first to mix the nickel compound with the Brønsted organic acid, and then next to introduce the aluminum compound. The molar ratio of the hydrocarbyl aluminum dihalide to the nickel compound, expressed by the Al/Ni ratio, is 2/1 to 50/1, and preferably 2/1 to 20/1. The molar ratio of the Brønsted acid to the nickel compound is 0.25/1 to 10/1, and preferably 0.25/1 to 5/1.

According to a preferred method, the hydrocarbyl aluminum dihalide can be enriched with an aluminum trihalide, the mixture of the two compounds then corresponding to the formula AlRₙX₃₋ₙ, in which R is a hydrocarbyl radical comprising 1 to 12 carbon atoms, such as alkyl, aryl, aralkyl, alkaryl or cycloalkyl, X is a chlorine or bromine atom, and n is a number between 0 and 1. Suitable mixtures include: dichloroethyl aluminum enriched with aluminum chloride, the mixture having a formula AlEt_{0.9}Cl_{2.1}; dichloroisobutyl aluminum enriched with aluminum chloride, the mixture having a formula AliBu_{0.9}Cl_{2.1}; and dibromoethyl aluminum enriched with aluminum bromide, the mixture having a formula AlEt_{0.9}Br_{2.1}.

The reaction for oligomerization of ethylene can be implemented at a temperature of −20 to 80 °C, preferably 40 to 60 °C, under pressure conditions such that the reagents are kept at least for the most part in the liquid phase or in the condensed phase. The pressure is generally between 0.5 and 5 MPa, preferably between 0.5 MPa and 3.5 MPa. The time of contact is generally between 0.5 and 20 hours, preferably between 1 and 15 hours.

The oligomerization stage can be implemented in a reactor with one or more reaction stages in a series, with the ethylene feedstock and/or the catalytic composition that is preferably pre-conditioned in advance being introduced continuously, either in the first stage, or in the first stage and any other one of the stages. At the outlet of the reactor, the catalyst can be deactivated, for example by injection of ammonia and/or an aqueous solution of soda and/or an aqueous solution of sulfuric acid. The unconverted olefins and alkanes that are optionally present in the feedstock are then separated from the oligomers by a separation stage, for example by distillation or washing cycles by means of caustic soda and/or water.

The conversion per pass is generally 85 to 98%. The selectivity of n-butenes that are formed is generally between 50 and 80%. The n-butenes consist of butene-2 (cis- and trans-) and butene-1.

The effluent generally contains less than 0.2% by weight of isobutene, or even less than 0.1% by weight of isobutene.

Separation of a Stream Rich in n-Butenes

The effluent that is obtained by dimerization of ethylene is subjected to a separation stage in such a way as to obtain an n-butene-enriched fraction.

The separation can be carried out by evaporation, distillation, extractive distillation, extraction by solvent or else by a combination of these techniques. These processes are known by one skilled in the art. Preferably, a separation of the effluent that is obtained by oligomerization of ethylene is carried out by distillation.

Preferably, the effluent of the oligomerization is sent into a distillation column system comprising one or more columns that makes it possible to separate, on the one hand, n-butenes from ethylene, which can be returned to the oligomerization reactor, and heavier olefins with 5 carbon atoms and more.

The higher olefins may subjected to hydrogenation so as to obtain renewably-sourced naphtha. “Renewably-sourced naphtha” shall mean naphtha produced from renewable sources. It is a hydrocarbon composition, consisting of mainly paraffins. The molecular weight of this renewably-sourced naphtha may range from hydrocarbons having 5 to 8 carbon atoms. Renewably-sourced naphtha can be used as a feedstock in steam-cracking to produce renewably-sourced light olefins, dienes and aromatics.

Hence, in an embodiment, step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractionating the effluent to recover a stream consisting essentially of n-butenes, a stream consisting essentially of heavier olefins, and an optional ethylene stream; and
subjecting the stream consisting essentially of heavier olefins to hydrogenation so as to obtain renewably-sourced naphtha.
   Metathesis of Ethylene with n-Butenes

Ethylene is able to undergo metathesis with n-butenes to produce propylene. In one aspect of the invention, step b)-(ii) comprises a metathesis reaction between n-butenes obtained according to step (i) and ethylene to obtain propylene. The n-butenes obtained according during ethylene dimerization (i) are a mixed stream including 1-butene and 2-butenes. Essentially only the 2-butenes react in a metathesis reaction, while 1-butene is essentially inert.

In one embodiment, 1-butene is removed from the mixed stream of 1-butene and 2-butenes and directed to a use elsewhere in the plant. Thus, in one embodiment, step b)-(ii) comprises removal of 1-butene from the mixed stream to obtain a stream rich in 2-butenes, and subjecting the stream rich in 2-butenes to the metathesis reaction. A stream rich in 2-butenes may comprise at least 90 wt.-% of 2-butenes, based on the total amount of n-butenes.

Alternatively, 1-butene may be converted to 2-butene by double bond isomerization. Double bond isomerization is an equilibrium-limited reaction. It is thus advantageous to subject the mixed stream of n-butenes to metathesis so as to react 2-butene with ethylene prior to double bond isomerization of 1-butene. Hence, in one embodiment the n-butenes are a mixed stream including 1-butene and 2-butenes, and b)-(ii) comprises
b)-(iia) subjecting the mixed stream to the metathesis reaction to obtain propylene and unreacted 1-butene;
b)-(iib) subjecting the unreacted 1-butenen to double bond isomerization to obtain 2-butenes; and
b)-(iic) recycling the 2-butenes obtained in step b)-(iib) to step b)-(iia).

In another embodiment, it is possible to convert 1-butene to 2-butene simultaneously with the metathesis reaction. For this purpose, a metathesis catalyst and an isomerization catalyst may be physically mixed or provided as distinct layers to allow both reactions to proceed simultaneously. Thus, in one embodiment, the n-butenes are a mixed stream including 1-butene and 2-butenes, and step b)-(ii) comprises passing the mixed stream through a metathesis/isomerization zone comprising both a metathesis catalyst and an isomerization catalyst. As 2-butene is consumed due to the metathesis reaction over the metathesis catalyst, it is thus replenished by isomerization of 1-butene to 2-butene over the isomerization catalyst.

The reaction is carried out in the presence of a metathesis catalyst on the basis of a metal which is selected from tungsten, molybdenum, rhenium, niobium, tantalum, vanadium, ruthenium, rhodium, iridium, osmium and nickel and the like. Tungsten, molybdenum and rhenium are preferred and tungsten is particularly preferred. Typically, tungsten catalysts are supported on silica, molybdenum and rhenium are supported on alumina based carriers. Especially preferred metathesis catalysts are WO₃-based catalysts, for example silica-supported WO₃ in the form of granules.

Suitable isomerization catalysts include magnesium-based catalysts such as MgO-based catalysts, for example tableted MgO.

Metathesis is carried out under conditions effective to produce an effluent comprising propylene, unconverted ethylene, and optionally 1-butene.

Unconverted ethylene and/or unconverted n-butenes may be recycled and combined with fresh ethylene and n-butenes to provided the metathesis feedstock.

The reaction may be conducted at 340-375 °C, 25-40 bar, a weight hourly space velocity (WHSV) of 7.5-30 hr⁻¹, and an ethylene to 2-butene molar ratio of 3:1 to 10:1.

The reactor effluent may be sent to a deethenizer to remove C₂ and lighter material. The bottoms from the deethenizer are sent to the depropenizer. High-purity, polymer-grade propylene (>99.9% molar purity) is recovered from the depropenizer overhead. The lighter material from the deethenizer and heavier C₄₊ material from the depropenizer are partly recycled to the reactors. Purge streams are provided for the lighter and heavier material to prevent buildup of inerts.

It should be noted that propane is not produced during the metathesis reaction. Consequently, polymer-grade propylene can be produced from the process, without the need for an expensive propylene-propane superfractionator.

Commercial processes for producing polymer-grade propylene by metathesis from ethylene and butenes feedstock are available from CB&I/Lummus (tradnemame OCT™) and from LyondellBasell.

### Oxidation of Propylene to Produce Acrolein or Acrylic Acid

In an aspect of the invention, the oxidation reaction is an oxidation reaction of propylene to produce an oxidation product selected from acrolein and acrylic acid.

It is common knowledge that acrylic acid can be produced by heterogeneously catalyzed gas phase oxidation of propylene with molecular oxygen over solid catalysts at temperatures between 200° to 400 °C in two stages via acrolein (cf. for example DE-A 19 62 431, DE-A 29 43 707, DE-C 1 205 502, EP-A 257 565, EP-A 253 409, DE-B 22 51 364, EPA 117 146, GB-C 1 450 986 and EP-A 293 224). The catalysts used are oxidic multicomponent catalysts based for example on oxides of the elements molybdenum, chromium, vanadium or tellurium. Five most commonly used catalyst systems for acrolein production are cuprous oxides, uranium antimony oxides, tin antimony oxides, bismuth molybdate oxides and multi-component bismuth molybdate based oxides. The most efficient catalysts for partial oxidation of propylene to acrolein consist of multi-component metal oxides systems. In almost every multi-component catalyst system, bismuth molybdate serves as the main ingredient. The following components are most commonly used as catalyst additives in molybdate bismuth oxide based catalysts: iron, cobalt, nickel, tungsten, potassium and phosphorous. Typical catalyst supports are inert porous solids, such as SiO₂, Al₂O₃, MgO, TiO₂, ZrO₂, aluminosilicates, zeolites, activated carbon, and ceramics.

The oxidation of propylene to acrylic acid can be carried out in one stage or two stages. Catalysts used for the heterogeneously catalyzed reaction are as a rule multimetal oxide materials which generally contain heavy metal molybdates as main component and compounds of various elements as promoters. The oxidation of propylene takes place in a first step to give acrolein and in a second step to give acrylic acid. Since the two oxidation steps may differ in their kinetics, uniform process conditions and a single catalyst do not as a rule lead to optimum selectivity. Recently, two-stage processes with optimum adaptation of catalyst and process variables have therefore preferably been developed. In general, propylene is oxidized to acrolein in the presence of molecular oxygen in the first stage in an exothermic reaction in a fixed-bed tubular reactor. The reaction products are passed directly into the second reactor and are further oxidized to acrylic acid. The reaction gases obtained in the second stage can be condensed and the acrylic acid can be isolated therefrom by extraction and/or distillation.

The oxidation of propylene to acrolein and/or acrylic acid is highly exothermic. The tubes of the fixed-bed tubular reactor which are filled with the heterogeneous catalyst are therefore surrounded by a cooling medium, as a rule a salt melt, such as a eutectic mixture of KNO₃ and NaNO₂. The heat of reaction is released through the wall of the catalyst-filled tubes to the salt bath.

Particularly preferred multimetal oxide materials have the formula I or II

[X¹ₐX²_{b}Oₓ]ₚ[X³_{c}X⁴_{d}X⁵ₑX⁶_{f}X⁷_{g}X²ₕO_{y}]_{q} (1)

Mo₁₂BiᵢX⁸ₖFeₗX⁹ₘX¹⁰ₙO_{z} (II)

where
X¹ is bismuth, tellurium, antimony, tin and/or copper, preferably bismuth,
X² is molybdenum and/or tungsten,
X³ is an alkali metal, thallium and/or samarium, preferably potassium,
X⁴ is an alkaline earth metal, nickel, cobalt, copper, manganese, zinc, tin, cadmium and/or mercury, preferably nickel and/or cobalt,
X⁵ is iron, chromium, cerium and/or vanadium, preferably iron,
X⁶ is phosphorus, arsenic, boron and/or antimony,
X⁷ is a rare earth metal, titanium, zirconium, niobium, tantalum, rhenium, ruthenium, rhodium, silver, gold, aluminum, gallium, indium, silicon, germanium, lead, thorium and/or uranium, preferably silicon, aluminum, titanium and/or zirconium,
a is from 0.01 to 8,
b is from 0.1 to 30,
c is from 0 to 4,
d is from 0 to 20,
e is from 0 to 20,
f is from 0 to 6,
g is from 0 to 15,
h is from 8 to 16,
x and y are numbers which are determined by the valency and frequency of the elements other than oxygen in I,
p and q are numbers whose ratio p/q is from 0.1 to 10,
X⁸ is cobalt and/or nickel, preferably cobalt,
X⁹ is silicon and/or aluminum, preferably silicon,
X¹⁰ is an alkali metal, preferably potassium, sodium, cesium and/or rubidium, in particular potassium,
i is from 0.1 to 2,
k is from 2 to 10,
l is from 0.5 to 10,
m is from 0 to 10,
n is from 0 to 0.5,
z is a number which is determined by the valency and frequency of the elements other than oxygen in II.

Multimetal oxide materials of the formula I are known per se from EP 0 000 835 and EP 0 575 897, and multimetal oxide materials of the formula II are known per se from DE 198 55 913.

Mostly, feeds for the oxidation contain a mixture of propylene, air, steam, and nitrogen. Steam and nitrogen are used to help control reactor hot-spot temperatures, and to provide a mixture which is not flammable. Beneficially, feed compositions range up to about 9 percent propylene on molar basis. Gaseous mixtures in the oxidation reactors are kept too low in oxygen to be flammable during normal operation. Reactor start-up and shutdown procedures are likewise designed to avoid flammable feed mixtures.

Briefly, a process for preparing acrylic acid typically comprises the steps of:
(a) catalytic gas phase oxidation of propylene and/or acrolein to acrylic acid to obtain a gaseous reaction product comprising acrylic acid;
(b) solvent absorption of the reaction product;
(c) distillation of the solvent loaded with reaction product in a column to obtain a crude acrylic acid and the solvent; and
(d) purification of the crude acrylic acid by crystallization.

Step (a) affords not pure acrylic acid, but a gaseous mixture which in addition to acrylic acid can substantially include unconverted acrolein and/or propylene, water vapor, carbon monoxide, carbon dioxide, nitrogen, oxygen, acetic acid, propionic acid, formaldehyde, further aldehydes and maleic anhydride.

The remaining, unabsorbed reaction gas of step (a) is further cooled down so that the condensable part of the low-boiling co-components thereof, especially water, formaldehyde and acetic acid, may be separated off by condensation. This condensate is known as acid water. The remaining gas stream, hereinafter called recycle gas, consists predominantly of nitrogen, carbon oxides and unconverted starting materials. Preferably, the recycle gas is partly recirculated into the reaction stages as diluting gas.

In commercial facilities the catalytic oxidation section typically consists of two tubular, fixed-bed reactors which are operated in series. Preferably the oxidation reactors are of the fixed-bed shell-and-tube type from about 3 to 5 meters long and about 1.9 to about 3.0 centimeters in diameter. Each reactor comprises between about 15,000 and about 35,000 tubes. The tubes are packed with catalyst, and optionally a small amount of inert material at the top serving as a preheater section for the feed gases.

The reactor tubes are cooled on the shell side by circulated a coolant which is typically a molten salt. Temperature of the coolant is controlled by heat exchangers, e.g., by circulating the molten salt through a steam generator which generates steam at elevated pressure. The steam is fed to the steam grid.

Vaporized propylene is mixed with steam and air and fed to the first-stage reactor wherein propylene is predominately converted to acrolein. The feed composition is typically from about 5 to about 7 percent of propylene, up to about 35 percent of steam, and the balance a gaseous source of dioxygen, typically compressed air or a mixture of compressed air and absorber vent gas.

The preheated gases react exothermically over the first-stage catalyst with the peak temperatures depending on conditions and catalyst selectivity. The conversion of propylene to oxides of carbon, i.e. carbon dioxide and carbon monoxide, is more exothermic than its conversion to acrolein. At the end of the catalyst bed the temperature of the mixture drops toward that of the molten salt coolant. Because acrolein at such elevated temperatures can undergo exothermic homogeneous decomposition to carbon oxides, i.e. carbon monoxide and carbon dioxide, first-stage reactor effluent is typically cooled to about 200° to about 250 °C in an interstage cooler to prevent homogeneous and non-selective oxidation of acrolein taking place in the pipes leading to the second-stage reactor.

Compressed air is preferably admixed with the first-stage reactor effluent upstream of second-stage oxidation reactor to provide oxygen for the oxidation reaction. The acrolein-rich gaseous mixture containing some acrylic acid is then passed to the second-stage reactor, which is similar to the first-stage reactor, but packed with a catalyst designed for selective conversion of acrolein to acrylic acid. The temperature of the effluent from the second-stage reactor again approximates that of the salt coolant. The heat of reaction is recovered as steam in external waste-heat boilers, and may be further cooled to about 220 °C and/or directly quenched.

Advantageously, the oxidations are operated at the lowest temperature consistent with high conversion. Conversion increases with temperature; the selectivity generally decreases only with large increases in temperature. Catalyst life also decreases with increasing temperatures. Catalysts are designed to give high performance over a range of operating conditions permitting gradual increase of salt temperature over the operating life of the catalysts to maintain productivity and selectivity near the initial levels, thus compensating for gradual loss of catalyst activity.

The oxidation of propylene to acrolein, as well as the oxidation of acrolein to acrylic acid, proceed with less than 100% selectivity and are accompanied by the combustion of propylene or acrolein over the catalyst, which gives carbon monoxide and carbon dioxide, herein collectively referred to as COₓ. It should be appreciated that emission of the carbon dioxide side product does not contribute to the carbon footprint of this process, as the starting propylene is carbon neutral.

Acrylic acid can be esterified in a conventional manner to the desired ester using the corresponding alcohol such as methanol, ethanol, n-propanol, isopropanol, n-butanol or 2-ethylhexanol, as described above with regard to the esterification of methacrylic acid to produce a methacrylic ester.

Acrylic acid can be polymerized, optionally together with one or more comonomers, to produce a water-absorbent resin. Water-absorbent resins are used to produce diapers, tampons, sanitary napkins and other hygiene articles, but also as water-retaining agents in market gardening.

A typical process for producing water-absorbent resins comprises polymerizing a monomer solution or suspension comprising
a) acrylic acid which may be at least partly neutralized,
b) at least one crosslinker,
c) at least one initiator,
d) optionally an ethylenically unsaturated monomer copolymerizable with acrylic acid, and
e) optionally one or more water-soluble polymers,
drying the resulting polymer gel, grinding the dried polymer gel, classifying and thermally surface postcrosslinking.

Suitable crosslinkers b) are compounds having at least two groups suitable for crosslinking. Crosslinkers b) are preferably compounds having at least two polymerizable groups which can be polymerized by free-radical polymerization into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane. With rising crosslinker content, the centrifuge retention capacity falls and the absorption under pressure passes through a maximum.

The initiators c) used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite.

To further improve the properties, the polymer particles can be surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides.

### Oxidation of Isobutene to Produce Methacrolein and Methacrylic Acid

In another aspect of the invention, the oxidation reaction is an oxidation reaction of isobutene to produce an intermediate selected from methacrolein and methacrylic acid.

Suitable oxidation catalysts for oxidizing isobutene to methacrolein are mixed metal oxide catalysts well known in the art.

A catalyst suitable for industrial production of methacrylic acid from isobutylene in high yield has not yet been found. Accordingly, it is industrially advantageous to conduct the reaction in two steps using a catalyst for production of methacrolein from isobutylene and a catalyst for production of methacrylic acid from methacrolein.

However, when methacrylic acid is produced in such a two step oxidation, it is relatively difficult to attain high selectivity of methacrylic acid in the second oxidation step if the conversion of methacrolein is high. That is, the selectivity of methacrylic acid formation decreases with increasing conversion of methacrolein. Accordingly, it becomes advantageous to conduct the second oxidation step under conditions such that the conversion of methacrolein is relatively low and then to recover the unreacted methacrolein from the reaction products and recycle them back into the second oxidation step. The unreacted methacrolein is separated from the reaction products in the second oxidation step while the object product, methacrylic acid is recovered. This is accomplished by cooling the reaction products in the second oxidation step to liquefy them, and/or further contacting them with water to separate the gaseous components of oxygen, nitrogen, carbon dioxide gas and the like. As a result, a liquid phase containing methacrolein and/or methacrylic acid, is obtained and these are separated by distillation.

### Epoxidation of Ethylene

In another aspect of the invention, the oxidation reaction is an epoxidation reaction of the renewably-sourced ethylene to produce ethylene oxide.

Ethylene oxide is produced in large volumes and is primarily used as an intermediate in the production of several industrial chemicals. Suitable epoxidation catalysts are generally obtained by depositing metallic silver on a support. Highly selective silver-based epoxidation catalysts have been developed, which comprise, in addition to silver as the active component, promoting species for improving the catalytic properties of the catalyst, as described in, e.g., WO 2007/122090 A2 and WO 2010/123856 A1. Examples of promoting species include alkali metal compounds and/or alkaline earth metal compounds, as well as transition metals such as rhenium, tungsten or molybdenum.

Suitable catalysts typically comprise 20 to 35% or 25 to 45 wt.-% of silver, relative to the weight of the catalyst.

As support material, it is possible to use various porous refractory materials such as activated carbon, titania, zirconia, silica, alumina or ceramic compositions or mixtures of these materials. Preferably, the refractory support is an aluminium oxide support. The supports preferably has a BET surface area of 0.5 to 3.0 m²/g.

A suitable catalyst may be obtained by
i) impregnating a refractory support with a silver impregnation solution, preferably under reduced pressure; and optionally subjecting the impregnated refractory support to drying; and
ii) subjecting the impregnated refractory support to a calcination process;
wherein steps i) and ii) are optionally repeated.

The epoxidation of ethylene preferably comprises reacting ethylene and oxygen in the presence of an epoxidation catalyst as described above. The epoxidation can be carried out by all processes known to those skilled in the art. It is possible to use all reactors which can be used in the ethylene oxide production processes of the prior art; for example externally cooled shell-and-tube reactors (cf. Ullmann's Encyclopedia of Industrial Chemistry, 5th edition, vol. A-10, pp. 117-135, 123-125, VCH-Verlagsgesellschaft, Weinheim 1987) or reactors having a loose catalyst bed and cooling tubes, for example the reactors described in DE 34 14 717 A1, EP 0 082 609 A1 and EP 0 339 748 A2.

The epoxidation is preferably carried out in at least one tube reactor, preferably in a shell-and-tube reactor. On a commercial scale, ethylene epoxidation is preferably carried out in a multi-tube reactor that contains several thousand tubes. The catalyst is filled into the tubes, which are placed in a shell that is filled with a coolant. In commercial applications, the internal tube diameter is typically in the range of 20 to 40 mm (see, e.g., U.S. Pat. No. 4,921,681 A) or more than 40 mm (see, e.g., WO 2006/102189 A1).

The coolant can be any of several well-known heat transfer fluids, such as tetralin (1,2,3,4-Tetrahydronaphthalene). The coolant exiting the reactor may be circulated through a steam generator which generates steam at elevated pressure. The steam is fed to the steam grid.

In reactors cooled with boiling water, the coolant is introduced to the cooling side of the reactor, most commonly the shell side of the reactor, as liquid water. As it flows through the cooling side, the water removes heat from the process side, and some of the water is vaporized to steam. The coolant exits the cooling side of the reactor as a mixture of water and steam. The steam exiting the reactor shell is removed and may be introduced in the steam grid. The temperature of the coolant in the reactor shell is determined by the boiling point of the water, which in turn is determined by the pressure under which it operates.

To prepare ethylene oxide from ethylene and oxygen, it is possible to carry out the reaction under conventional reaction conditions as described, e.g., in DE 25 21 906 A, EP 0 014 457 A2, DE 23 00 512 A1, EP 0 172 565 A2, DE 24 54 972 A1, EP 0 357 293 A1, EP 0 266 015 A1, EP 0 085 237 A1, EP 0 082 609 A1 and EP 0 339 748 A2. Inert gases such as nitrogen or gases which are inert under the reaction conditions, e.g. steam, methane, and also optionally reaction moderators, for example halogenated hydrocarbons such as ethyl chloride, vinyl chloride or 1,2-dichloroethane can additionally be mixed into the reaction gas comprising ethylene and molecular oxygen.

The reaction gas preferably comprises a chlorine-comprising reaction moderator such as ethyl chloride, vinyl chloride or 1,2-dichloroethane in an amount of from 0 to 15 ppm by weight, preferably in an amount of from 0.1 to 8 ppm by weight, based on the total weight of the reaction gas. The remainder of the reaction gas generally comprises hydrocarbons such as methane and also inert gases such as nitrogen. In addition, other materials such as steam, carbon dioxide or noble gases can also be comprised in the reaction gas.

The concentration of carbon dioxide in the feed (i.e. the gas mixture fed to the reactor) typically depends on the catalyst selectivity and the efficiency of the carbon dioxide removal equipment. Carbon dioxide concentration in the feed is preferably at most 3 vol.-%, more preferably less than 2 vol.-%, most preferably less than 1 vol.-%, relative to the total volume of the feed. An example of carbon dioxide removal equipment is provided in U.S. Pat. No. 6,452,027 B1.

The epoxidation of ethylene to ethylene oxide is usually carried out at elevated catalyst temperatures. Preference is given to catalyst temperatures in the range of 150 to 350 °C, more preferably 180 to 300 °C, particularly preferably 190 to 280 °C and especially preferably 200 to 280 °C.

The epoxidation is preferably carried out at pressures in the range of 5 to 30 bar. All pressures herein are absolute pressures, unless noted otherwise. The epoxidation is more preferably carried out at a pressure in the range of 5 to 25 bar, such as 10 bar to 24 bar and in particular 14 bar to 23 bar.

The epoxidation of ethylene is preferably carried out under conditions conducive to obtain a reaction mixture containing at least 2.3 vol.-% of ethylene oxide. In other words, the ethylene oxide outlet concentration (ethylene oxide concentration at the reactor outlet) is preferably at least 2.3 vol.-%. The ethylene oxide outlet concentration is more preferably in the range of 2.5 to 4.0 vol.-%, most preferably in the range of 2.7 to 3.5 vol.-%.

The epoxidation is preferably carried out in a continuous process. The epoxidation of ethylene can advantageously be carried out in a recycle process. After each pass, the newly formed ethylene oxide and the by-products formed in the reaction are removed from the product gas stream. The remaining gas stream is supplemented with the required amounts of ethylene, oxygen and reaction moderators and reintroduced into the reactor. The separation of the ethylene oxide from the product gas stream and its work-up can be carried out by customary methods of the prior art (cf. Ullmann's Encyclopedia of Industrial Chemistry, 5th edition, vol. A-10, pp. 117-135, 123-125, VCH-Verlagsgesellschaft, Weinheim 1987).

The epoxidation of ethylene proceeds with less than 100% selectivity and is accompanied by the generation of carbon dioxide. It should be appreciated that emission of the carbon dioxide side product does not contribute to the carbon footprint of this process, as the starting ethylene is carbon neutral.

The ethylene oxide may be subjected to a hydrolysis reaction to produce ethylene glycol, or else an amination reaction of the ethylene oxide to produce ethanolamines and/or ethylene amines.

The invention is further illustrated by the example that follows.

### Example (Prophetical)

The effects of the invention are illustrated with respect to an exemplary embodiment of acrylic acid manufacture.

The conversion of 0.7 t of propylene to 1 t of acrylic acid using standard catalysts produces a total of 0.35 t of CO₂ as a yield loss. The cooling of the exothermic reaction zones of the process generates 2.3 t of heated steam from steam condensate.

To generate this amount of heated steam, 0.14 t of bio-ethanol would have to be burned. This amount of bio-ethanol can be saved by interconnecting ethanol dehydration and/or olefin interconversion with the acrylic acid production according to the invention. Put otherwise, the combustion of part of the renewably-sourced ethanol to generate heat is shifted to a later production step, namely the production of acrylic acid, as part of the inevitable yield losses there. The steam requirement for producing 0.7 t renewably-sourced propene from renewably-sourced ethanol by the sequence of ethanol dehydration, ethylene dimerization to n-butenes, metathesis reaction between n-butenes and ethylene to propylene is in the range of from 2.2 to 3.0 t steam. Thus, the amount of heated steam generated by heat recovery from the acrylic acid manufacture can in principal cover the steam requirement of the ethanol to olefin conversion completely or at least to a large extent.

1.-10. (canceled)

11. A process for the manufacture of acrylic acid in combination with generation of heated steam, said process comprising the steps of:
a) subjecting a feedstock comprising a renewably-sourced ethanol to dehydration to produce a renewably-sourced ethylene stream;
b) subjecting the renewably-sourced ethylene stream to an olefin-interconversion to obtain a renewably-sourced propylene; the olefin-interconversion comprising (i) and (ii):
(i) ethylene dimerization to obtain n-butenes;
(ii) metathesis reaction between n-butenes obtained according to (i) and ethylene to obtain propylene; and
c) subjecting the renewably-sourced propylene to an oxidation reaction to obtain acrylic acid; and
removing at least a portion of heat generated by the oxidation reaction and transferring at least a portion of the heat to a water stream to generate a heated steam stream.

12. The process according to claim 11, wherein the heated steam stream is used to provide heat to a heat-accepting process.

13. The process according to claim 12, wherein the heated steam stream is used to provide at least a portion of the heat consumed in at least one of steps a) and b).

14. The process according to claim 11, wherein the oxidation reaction is accompanied by over-oxidation of propylene to carbon dioxide.

15. The process according to claim 14, wherein 1 to 10 mol.-% of the propylene is over-oxidized to carbon dioxide.

16. The process according to claim 14, wherein the amount of biogenic CO₂ released in the oxidation reaction is in the range from 50 to 100 wt.-%, based on the total amount of CO₂.released in the oxidation reaction.

17. The process according to claim 11, wherein step b)-(i) comprises:
contacting the renewably-sourced ethylene stream with a dimerization catalyst in a dimerization zone;
operating said dimerization zone at conditions effective to produce an effluent consisting essentially of n-butenes, a stream consisting essentially of heavier olefins, and optionally an unconverted ethylene stream;
fractioning the effluent to recover a stream consisting essentially of n-butenes, a stream consisting essentially of heavier olefins, and an optional ethylene stream; and
subjecting the stream consisting essentially of heavier olefins to hydrogenation so as to obtain renewably-sourced naphtha.

18. The process according to claim 11, wherein the n-butenes are a mixed stream including 1-butene and 2-butenes, and wherein b)-(ii) comprises removal of 1-butene from the mixed stream to obtain a stream rich in 2-butenes, and subjecting the stream rich in 2-butenes to the metathesis reaction.

19. The process according to claim 11, wherein the n-butenes are a mixed stream including 1-butene and 2-butenes, and wherein b)-(ii) comprises
b)-(iia) subjecting the mixed stream to the metathesis reaction to obtain propylene and unreacted 1-butene;
b)-(iib) subjecting the unreacted 1-butene to double bond isomerization to obtain 2-butenes; and
b)-(iic) recycling the 2-butenes obtained in step b)-(iib) to step b)-(iia).

20. The process according to claim 11, wherein the n-butenes are a mixed stream including 1-butene and 2-butenes, and wherein b)-(ii) comprises passing the mixed stream through a metathesis/isomerization zone comprising both a metathesis catalyst and an isomerization catalyst.
